(19) United States
(12) Patent Application Publication
(10) Pub. No.: US 2002/0019650 A1
(43) Pub. Date: Feb. 14, 2002

(54) Method of controlling spasticity by implants in spinal cord injury

(76) Inventors: Michael Craggs, London (GB); Sarah Louise Knight, Hertfordshire (GB)

Correspondence Address: William C. Rowland, BURNS, DOANE,, SWECKER & MATHIS, L.L.P., P.O. Box 1404, Alexandria, VA, 22313-1404, US

(21) Appl. No.: 09/897,052

(22) Filed: Jul. 3, 2001

Related U.S. Application Data
(60) Provisional application No. 60/216,371, filed on Jul. 5, 2000.

(51) Int. Cl. A61N 1/36

(52) U.S. Cl. 607/2

(57) ABSTRACT

A method of controlling spasticity in patients having spinal cord injury (SCI) includes applying electrical stimuli to the sensory and motor sacral nerve pathways at one or more of the S1 to S4 levels by electrodes connected directly to the respective nerve roots and monitoring the resulting muscle activity.

This invention relates to a method and apparatus for controlling lower limb spasticity by implants in spinal cord injury.

Spasticity is a common and distressing manifestation of neurological damage resulting from spinal cord injury (SCI) and leads to violent lower limb spasms. There are about 700 new spinal injuries in Britain each year, with a total population of sufferers of about 40,000. Injuries of this kind are commonly caused by road accidents and of new patients, the greatest proportion are young people who, with good care and management, can expect to live a normal life span. Therefore, to enable these young people to reintegrate into society and have a good quality of life and reach their full potential, there is a need for a new and innovative method for restoring functions.

Following SCI, the normal ascending and descending neural pathways in the spinal cord are interrupted and after the initial period of shock, a number of aberrant reflexes emerge. These reflexes can lead to spasticity of muscle groups including the skeletal muscles of the limbs, as well as the pelvic sphincters and the smooth muscle of the bladder and bowel. Spasticity can be defined as an increase in muscle tone which is characterised by abnormal muscle relaxation, hyper-reflexia and flexor and extensor spasms or involuntary contractions of the paralysed limbs. Muscular spasm adversely effects the quality of life of the SCI patient, and can be a severe restriction to rehabilitation.

The current treatments for spasticity and muscular spasm include pharmacological, surgical and physiotherapy techniques. Pharmacological agents operate by relaxing muscle at the central nervous system level. However, the drugs which operate in this way are associated with side-effects which cause drowsiness and fatigue. Surgical techniques include surgical section of the sensory nerve roots at the sacral or lumbar level of the spine. Neural surgery, however, removes the possibility of recovery of function associated with such known pathways. Also, even where restoration of any sensory perception is unlikely, preservation of sensory nerve pathways reduces muscle wastage in the lower limbs and helps to prevent development of pressure sores which can be very debilitating.

The present invention is directed to a solution to the above problems which utilise electrical stimulation of roots of specified nerves by a procedure known as neuromodulation.

According to one aspect of the present invention there is provided a method of controlling spasticity in patients having spinal cord injury (SCI) which comprises applying electrical stimuli to the sensory and motor sacral nerve pathways at one or more of the lumbar-sacral levels of the spine, especially the S2 to S4 levels.

The electrical stimulation is applied generally by means of invasive electrodes maintained in the proximity of the spinal nerve roots. This can be achieved by physically attaching the electrodes to the nerve roots, e.g. with loops of insulated material or surrounding the roots with an insulating sheath within which the electrode is secured in contact with the nerve.

The electrodes are held in close proximity to both the sensory and motor sacral nerve pathways at one or more of the lumbar and/or sacral levels. The electrodes may be extradural or intrathecal (i.e. within or outside the spinal canal). The electrical stimulation may be applied using a Finetech-Brindley electrical stimulator, which is commercially available from Finetech Medical Limited, Welwyn Garden City, England.

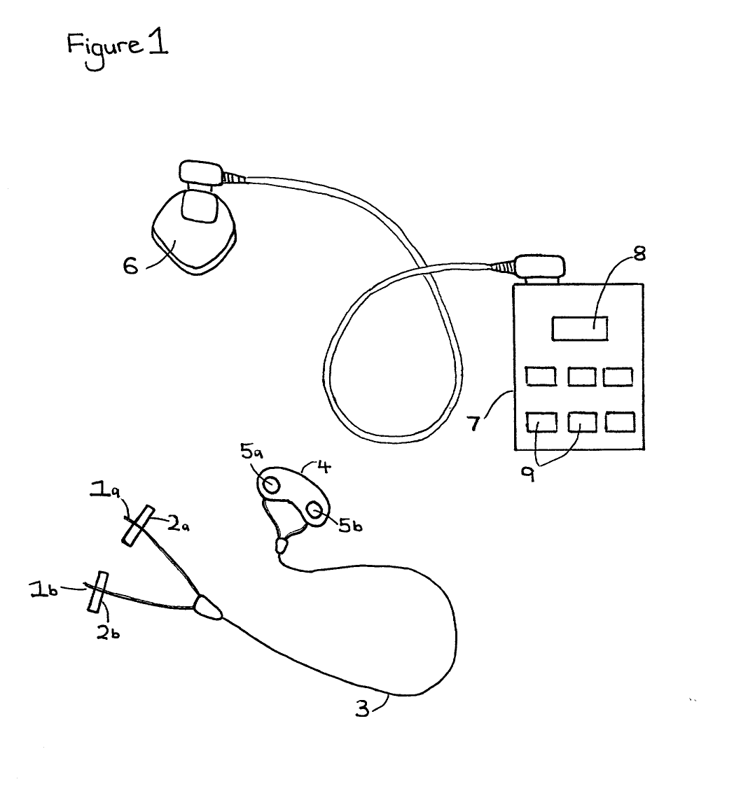

Such stimulator comprises a radio transmitter which is worn externally of the patient connected to an external transmitter coil, which is worn by the patient usually in the area of the chest or abdomen and an implanted receiver attached to the implanted electrodes as described above. The implanted receiver comprises an tuned circuit which responds to the modulated radio signals produced by the external transmitter. The equipment is shown diagrammatically in FIG. 1, in which 1a and 1b show the electrodes which are in contact with the sacral and/or lumber anterior and posterior nerve roots. For example, the electrodes can be held in contact with the anterior and posterior nerve roots by means of silicone loops 2a and 2b. Electrodes 1a and 1b are connected by a catheter 3 to a housing 4 for two or more radio receivers 5a, 5b, for generating electrical stimuli for each of the electrodes. Housing 4 comprises a casing of insulating material which is inert to body fluids and into which the receivers are sealed Receivers 5a and 5b are passive receivers requiring no power source of their own and are activated by an external transmitter 6 including a transmitter coil (not shown). Transmitter 6 is typically worn on the patient's chest or abdomen e.g. as a harness. The transmitter is controlled by a control unit 7, which includes an LED screen 8 and buttons 9 for adjusting parameters such as amplitude (voltage), frequency, pulse shape and duration and ramping (time to reach maximum frequency and/or amplitude.

A laminectomy operation is required to introduce the electrodes in the lumbar-sacral area and during the operation, the electrodes are preferably physically attached to the anterior and posterior nerve roots, e.g. with silicone loops attached to the electrodes. Some degree of individual adjustment may be necessary to establish the most effective pattern of electrical stimulation to the nerve roots. This may involve varying the pattern of neuromodulation applied to the sensory motor nerves using the control unit 7 after provoking a lower limb spasm and noting the speed by which the spasm is suppressed under different values of the above parameters.

The equipment employed for producing the electrical signal is similar to that described in U.S. Pat. No. 3,870,051 (the disclosure of which is hereby incorporated specifically by reference).

The electrode terminal devices themselves are described in U.S. Pat. No. 3,718,134 which is also specifically incorporated hereby by reference.

It has been found that the levels of stimulation required to modulate and control spasticity in the lower limbs is substantially less than that needed in bladder control. For example, the method of the present invention works well at a frequency of less than 20 pulses/second, preferably about 10 to 15 pulses/second. Also, the pulse width tends to be considerably shorter, e.g. about ⅕ or ⅙ of that required for bladder control It has also been found that it is advantageous to ramp the signal more gradually up to the threshold level determined by empirical tests with a particular patient.

It is also possible to use the same implanted electrodes and control unit to control bladder, sphincter and bowel contractions and to maintain the patient in a continent condition. This can be done by programming into the control unit a set of background levels of the above parameters to control spasm while applying an overlay programme for controlling bladder and bowel operation.

1. A method of controlling spasticity in patients having spinal cord injury (SCI) which comprises applying electrical stimuli to the sensory and motor sacral nerve pathways at one or more of the lumbar-sacral levels.

2. A method according to claim 1 wherein the electrical stimuli are applied by means of invasive electrodes maintained in the proximity of the lumbar-sacral nerve roots.

3. A method according to claim 2 wherein the electrodes are held in contact with the sacral nerve roots.

4. A method of controlling spasticity in patients having spinal cord injury (SCI) which comprises applying electrical stimuli to the sensory and motor sacral nerve pathways at one or more of the S1 to S4 levels by means of electrodes connected directly to the respective nerve roots and monitoring the resulting muscle activity.

5. A method according to claim 4 wherein the nerve roots are stimulated by a varying pattern of frequency, amplitude, pulse width and pulse shape.
